(19) United States
(12) Patent Application Publication
(10) Pub. No.: US 2009/0024158 A1
(43) Pub. Date: Jan. 22, 2009

(54) Access Port Expander And Method

(75) Inventor: Thomas O. Viker, Arden Hills, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/778,123

(22) Filed: Jul. 16, 2007

(51) Int. Cl. A61B 17/00; A61B 1/32

(52) U.S. Cl. 606/201; 600/208

(57) ABSTRACT

A surgical access port expander for expanding an access path between an incision site and a surgical site during a surgical procedure. The access port expander has an actuator with an actuator rod connected to a plurality of blades. The blades are movable in response to motion of the actuator rod moving in a direction substantially parallel to the blade lengths. Further, each blade is moved in a direction substantially perpendicular to its respective length from a contracted position to an expanded position.

The present application is related to “Surgical Site Access System and Deployment Device for Same” (Attorney Docket No. ZMS-MI03US) filed on even date herewith, the disclosure of which is hereby incorporated herein by reference in its entirety.

### FIELD OF THE INVENTION

The present invention generally relates to access systems useful in various surgical procedures, and more particularly to an improved access system useful for minimally invasive surgical procedures.

### BACKGROUND OF THE INVENTION

In the past, certain surgical procedures required relatively large incisions to be made into the body in order to gain visual and instrument access to a surgical site deep within the body. By way of example, surgical procedures on posterior spine elements may have required relatively large incisions to be made to effectively operate on the spine elements. These large incisions are generally undesirable as they may result in increased damage to muscle tissue, increased blood loss, prolonged pain to the patient as well as potential scarring.

More recently, however, many surgical procedures are conducted using minimally invasive techniques that seek to minimize some of the undesirable aspects of past procedures by creating a relatively small incision and then increasing the effective size of the opening using various dilators and/or retractors. Dilation, in effect, splits the muscle tissue, as opposed to cutting the muscle tissue, which in turn causes less damage to the muscle, increases the recovery times, and reduces patient discomfort. Retraction is used to hold the incision and passageway open through the soft tissue and muscle.

Once the incision is dilated and a path to the surgical site is established, an access port may be inserted through the incision to provide the necessary retraction so as to establish an unencumbered path to the surgical site that effectively defines a working channel or space. The access port provides visible access and instrument access to the surgical site in a minimally invasive manner.

Accordingly, there is a need for an improved access system and method of using the same that addresses drawbacks of current access ports and associated procedures.

### SUMMARY OF THE INVENTION

The present invention overcomes the foregoing and other shortcomings and drawbacks of access port systems heretofore known. While the invention will be described in connection with certain embodiments, it will be understood that the invention is not limited to these embodiments. On the contrary, the invention includes all alternatives, modifications and equivalents as may be included within the spirit and scope of the present invention.

An improved access port system that includes an access port expander for expanding an access port to create an access path between an incision site and a surgical site during a surgical procedure. The access port expander of the present invention is effective to radially spread an access port, thus spreading and separating tissue laterally with respect to the incision site. Such a lateral spreading minimizes cutting of tissue and is generally parallel to a major surface of the skin, which tends to minimize and reduce wound trauma. Further, such a lateral spreading minimizes separating of tissue in a direction generally perpendicular to a major surface of the skin, which may further reduce wound trauma. The access port expander of the present invention may be used in performing spinal surgical procedures.

According to the principles of the present invention and in accordance with the described embodiments, the invention provides a surgical access port expander for expanding an access path between an incision site and a surgical site during a surgical procedure. The access port expander has an actuator with an actuator rod connected to a plurality of blades. The blades are movable in response to motion of the actuator rod moving in a direction substantially parallel to the blade lengths. Further, each blade is moved in a direction substantially perpendicular to its respective length from a contracted position to an expanded position.

In another embodiment, the invention provides a method of using an access port expander during a surgical procedure to expand an access path between an incision site and a surgical site in a patient. A contracted access port expander is inserted through a collapsed expandable access port. An incision is made to create an incision site, and the contracted access port expander and the collapsed expandable access port are inserted through the incision site until one end of the collapsed expandable access port is near the surgical site. An actuator on the access port expander is operated to move an actuator rod in a first direction substantially parallel to the lengths of access port expander blades. Simultaneously, the blades are moved by the actuator rod in a second direction substantially perpendicular to the blade lengths. Thus, the blades expand radially outward to cause the access port to simultaneously expand outward in the second direction to an expanded state. Thereafter, the access port expander is removed from the expanded access port to provide an expanded access path between the incision site and the surgical site to facilitate a surgical procedure.

These and other objects, advantages and features of the invention will become more readily apparent to those of ordinary skill in the art upon review of the following detailed description taken in conjunction with the accompanying drawings.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1A is a perspective view of an exemplary embodiment of an access port system with an access port expander shown in a contracted state in accordance with the principles of the present invention.

FIG. 1B is a perspective view of the exemplary access port system of FIG. 1A in which the access port expander is shown in an expanded state in accordance with the principles of the present invention.

FIGS. 1C and 1D are other perspective views of the access port expander of FIG. 1A shown respectively in a contracted state and an expanded state.

FIG. 2A is a cross-sectional view of one embodiment of a mechanism used to operate the access port expander of FIGS. 1A and 1B.

FIG. 2B is an exploded view of a portion of the access port expander mechanism shown in FIG. 2A in which the mechanism undeployed.

FIG. 2C is an exploded view of the portion of the access port expander mechanism shown in FIG. 2B in which the mechanism is deployed.

FIG. 3 is a cross-sectional view schematically illustrating another exemplary embodiment of a mechanism used to operate the access port expander of FIGS. 1A and 1B.

FIG. 4 is a cross-sectional view schematically illustrating a further exemplary embodiment of a mechanism used to operate the access port expander of FIGS. 1A and 1B.

FIG. 5 is a cross-sectional view schematically illustrating a still further exemplary embodiment of a mechanism used to operate the access port expander of FIGS. 1A and 1B.

FIGS. 6A and 6B are perspective views schematically illustrating yet further exemplary embodiments the access port expander of FIGS. 1A and 1B.

### DETAILED DESCRIPTION OF THE PREFERRED EMBODIMENTS

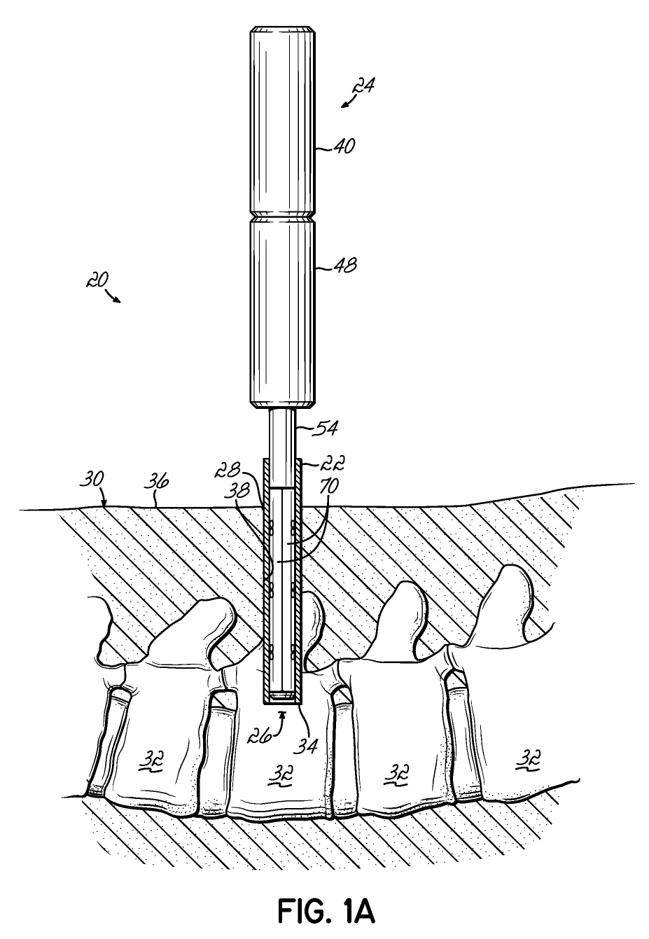

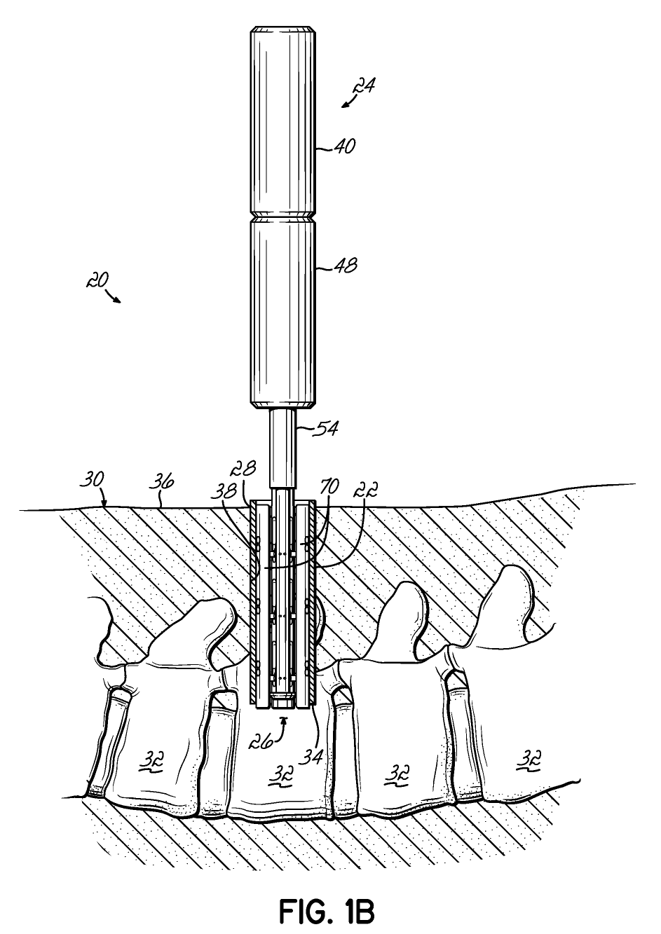

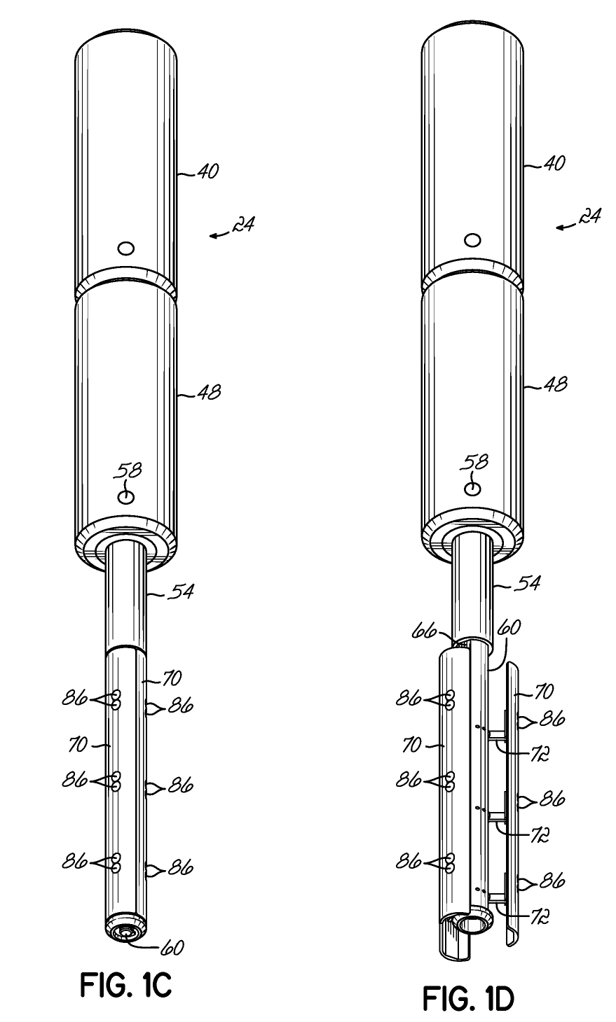

Referring now to the figures, and to FIG. 1A in particular, a surgical access system 20 according to the principles of the present invention includes an access port 22 and an access port expander 24 extending through the access port 22. In various exemplary embodiments, the access port 22 may be made of, for example, an expandable material, such as plastics, flexible metal materials, or a braided material such as a braided wire mesh. The surgical access system 20 may be used during a minimally invasive surgical procedure to expose and provide visual and instrument access to a surgical site 26 that is spaced from an incision site 28 on a patient 30. In this exemplary embodiment, the access port system 20 is illustrated as being used for posterior spine surgery, wherein certain spinal elements 32 are located at the surgical site 26 and are spaced from an associated incision site 28 along the back of a patient 30. While the access port 22 and access port expander 24 are shown in the context of posterior spinal surgery, they may be used in a wide variety of surgical procedures where the surgical site is spaced from the incision site and unencumbered access to the surgical site via the incision site is desired.

Generally, in use, an access port expander 24 in its contracted state is inserted into an expandable access port 22 that is in its collapsed state. This may be done before or after an incision site 28 is created. A small preliminary path or opening is created from the incision site to a surgical site 26 using a pointed instrument, a K-wire or other comparable device in a known manner. Thereafter, the assembly of the contracted access port expander 24 and the collapsed access port 22 is inserted through the incision site 28 until a lower end 34 of the collapsed access port 22 is in the proximity of the surgical site 26. The access port expander 24 is than deployed radially outward, that is, in a direction generally perpendicular to a length of the access port expander 24. This outward expansion of the access port expander 24 causes an expansion of the access port 22 in the same direction, that is, generally perpendicular to the length of the access port expander 24 as shown in FIG. 1B. Such an expansion of the access port 22 dilates an opening 38 in the tissue. Thereafter, the access port expander 24 is removed from the access port 22, thereby permitting a visual and instrument access to the surgical site 26 through the expanded access port 22.

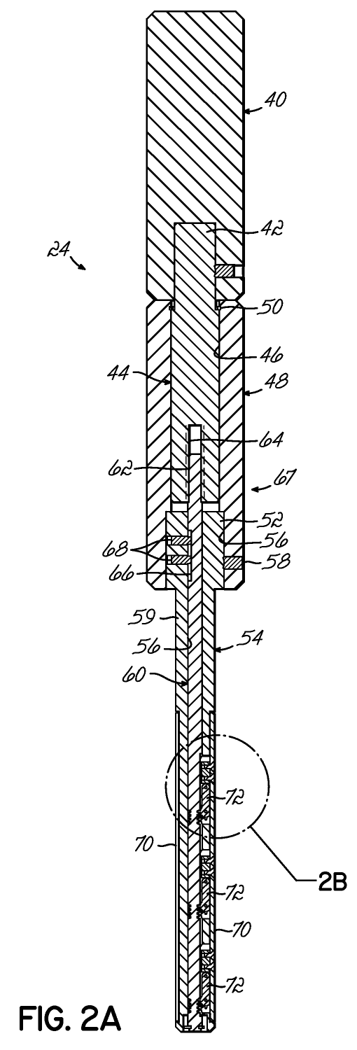

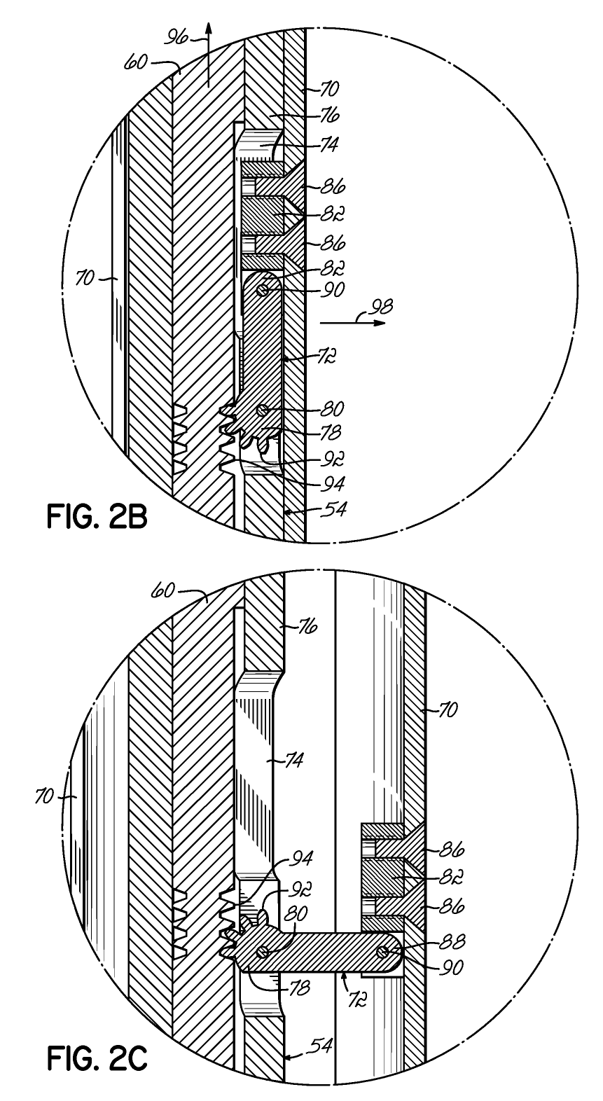

Referring to FIGS. 1C and 1D, the access port expander 24 is further shown with its blades 70 in their respective contracted and expanded states. One embodiment of a mechanism for operating the access port expander 24 is shown in FIG. 2A. An upper handle 40 is rigidly connected to one end 42 of a drive screw 44 that is rotationally mounted within a center bore 46 of a lower handle 48. The upper handle 40 is longitudinally fixed with respect to the lower handle 48 by a bearing 50 that has an inner race press fit onto the drive screw 44 and an outer race press fit into the center bore 46. Thus the upper handle 40 may be turned or rotated with respect to the lower handle 48, but the upper handle 40 cannot be removed or separated from the lower handle 48.

An upper end 52 of a cylindrical, tubular frame 54 is mounted in an end bore 56 of the lower handle 48 and nonrotatably fixed therein by a pin, screw or comparable fastener 58. Thus the frame 54 and lower handle 48 are connected together as a unitary structure. The frame upper end 52 is larger to better secure it in the end bore 56; however, the main body 59 of the frame 54 is a lesser diameter to facilitate its insertion through an incision site. An actuator rod 60 has a threaded end 62 engaged with a threaded and centered end bore 64 of the drive screw 44. Further, the actuator rod 60 extends through a center bore of 61 of the frame 54. The actuator rod 60 has a flat or land 66 extending longitudinally on its outer surface. Anti-torque guide pins 68 extend through the frame 54 such that their ends are immediately adjacent to the flat 66, thereby preventing the actuator rod 60 from rotating but permitting the actuator rod 60 to move lengthwise. If, for example, the threaded connection between the drive screw 44 and rack 60 is a right-hand thread, a clockwise rotation of the upper handle 40 and drive screw 44 results in the actuator rod 60 moving upward as viewed in FIG. 2A. Thus, the upper handle 40, drive screw, 44, lower handle 48, frame 54 and actuator rod 60 function together as an actuator 67 for the access port expander 24.

Three blades 70 are moveably mounted to the frame 54 and substantially surround the actuator rod 60. A mounting structure that is commonly used in this embodiment is illustrated in detail in FIG. 2B. A drive link 72 is located within an opening 74 in a wall 76 of the frame 54. A drive link pinion end 78 is pivotally mounted to the frame 54 by a pivot pin 80 extending through the frame wall 76 and the pinion end 78. A bearing block 82 is mounted against an interior surface 84 of the frame wall 76 by screws 86 or other appropriate fasteners. A drive end 88 of the drive link 72 is pivotably mounted to the bearing block 82 by a pivot pin 90 that is fixed in the bearing block 82.

The pinion end 78 has teeth 92 that mesh with rack teeth 94 on the actuator rod 60. Assume, in this exemplary embodiment, that the actuator rod threaded end 62 is threaded into the drive screw end bore 64 with a right-hand thread. Therefore, as the top handle 40 is turned clockwise, the actuator rod 60 is moved axially upward as indicated by the arrow 96 of FIG. 2B; and the pinion ends 78 and respective drive links 72 are moved generally clockwise. Clockwise rotation of the drive links 72 causes the blades 70 to move outward in a generally radial direction with respect to the actuator rod 60 as indicated by the arrow 98. Thus, in response to the actuator rod 60 moving in a direction generally parallel to the blade lengths, each blade 70 move in a direction generally perpendicular to a respective blade length to a fully expanded position as shown in FIG. 2C. Further, referring to FIG. 1B, as the blades 70 are moved radially outward, that is, substantially perpendicular to axial centerline of the actuator rod 60, they are effective to push on an interior surface of the access port 22 causing it to radially expand to a larger diameter or size, thereby dilating the opening 38. In an alternative embodiment, the actuator rod threaded end 62 may be threaded into the drive screw end bore 64 with a left-hand thread; and in that embodiment, the actuator rod 60 is moved axially upward by a counter-clockwise rotation of the top handle 40.

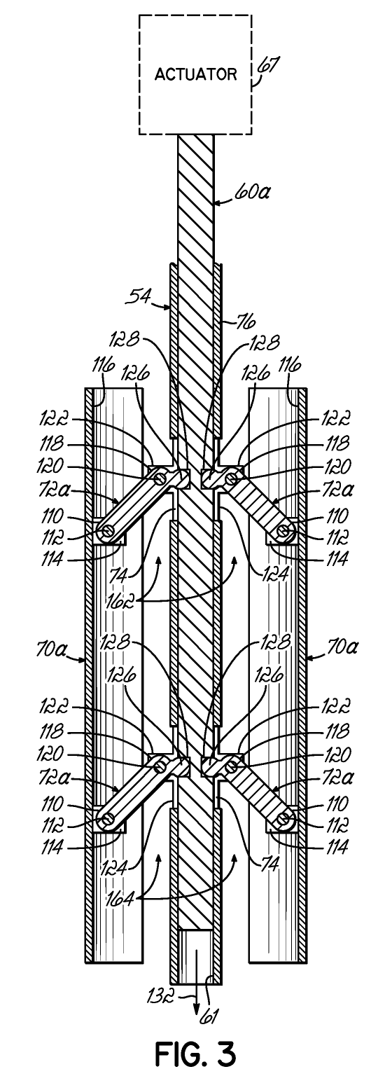

Another exemplary embodiment of a mechanism for spreading the blades 70 of the access port expander 24 is schematically shown in FIG. 3. Referring to FIG. 3, an actuator rod 60a replaces the actuator rod 60 previously described with respect to FIG. 2A. The actuator rod 60a is mounted for lengthwise sliding motion inside the threaded end bore 62 of the drive screw 44 in a manner substantially similar to that described with respect to FIG. 2A. However, in the embodiment of FIG. 3, links 72a are used to drive the blades 70a radially outward with respect to the actuator rod 60a. Each link 72a has one end 110 that is pivotally mounted to a pivot pin 112 that, in turn, is supported by a bearing block 114 mounted to an interior surface 116 of a blade 70B. An opposite end 118 of the link 72a is pivotally mounted on a pivot pin 120 that is fixed on a pivot block 122 extending from an outer surface 124 of frame wall 76. Each link 72a further has a crank arm 126 having a curved or spherical profile that permits the crank arm 126 to be pivotally mounted within a cavity or hole 128 in an outer surface of the actuator rod 60a.

As previously described with respect to the access port expander 24 of FIG. 2A, the actuator rod 60a of FIG. 3 has a threaded upper end that threadedly engages a drive screw 44 rotationally mounted in the lower handle 48 and rigidly connected to the upper handle 40. Thus, in a manner as previously described, rotation of the upper handle 40, for example, in a counter-clockwise direction, moves the actuator rod 60a in a downward direction as indicated by the arrow 132 in FIG. 3. Moving the actuator rod 60a downward also pushes the crank arms 126 downward and causes a counter-clockwise rotation of respective links 72a, thereby moving the blades 70a in a radially outward direction with respect to the actuator rod 60a.

In a manner as previously described, the motion of the blades 70a in a direction substantially perpendicular to an actuator rod centerline, or the blade lengths, can be used to radially expand an access port. In the embodiment of FIG. 3, two blades 70a are shown. If each of the blades 70a has a substantially semicircular cross-sectional profile, then a radially outward motion of the blades 70a creates an access port that has a generally elongated cross-sectional profile.

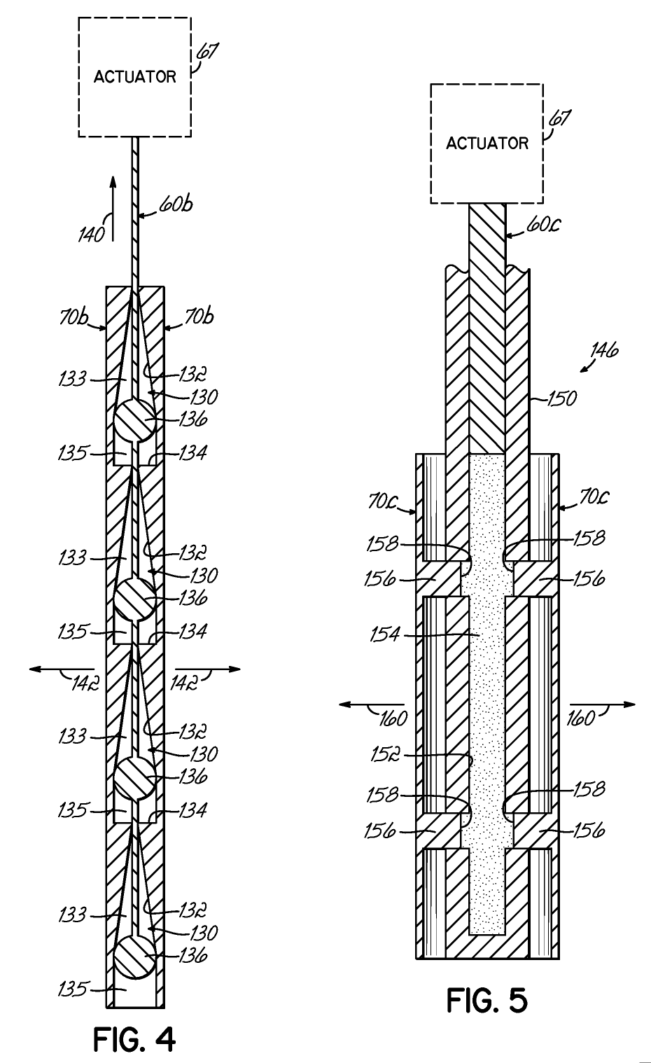

Referring to FIG. 4 a further embodiment of a mechanism for operating an access port expander is shown. In this embodiment, each of the blades 70b has a respective inner cavity 130 formed by respective inner surfaces 132, 134. Inner surface 134 extends generally radially outward from the actuator rod 60b, and inner surface 132 extends lengthwise and is angled inward back toward the actuator rod 60a. Opposed pairs of inner cavities 130 of the blades 70b form a volume or cavity having a generally conical upper end 133 and a generally cylindrical lower end 135. Each pair of opposed tapered cavities 130 has a respective spreader ball 136 located therein. When the spreader ball 136 is located in a lower cylindrical cavity 135, the blades are in close proximity; and the access port expander is in a closed state. All of the spreader balls 136 are connected to the actuator rod 60b that extends longitudinally between the blades 70b. In a manner similar as previously described, the upper end of the actuator rod 60b is threaded into an end bore of a drive shaft of 44 shown in FIG. 2A and hence, is part of an actuator 67 the operation of which has been previously described. Thus, rotation of the upper handle 40 and drive bore 44 with respect to the lower handle 48 operates to move the actuator rod 60b and spreader balls 136 in a upward direction as indicated by the arrow 140 in FIG. 4. The upward motion of the spreader balls 136 forces the blades 70b to be displaced in a radially outward direction as indicated by the arrow 142. Translating the blades in a direction perpendicular to an actuator rod centerline, or the blade lengths, is effective to radially expand an access port as previously described. The illustration of the exemplary embodiment of FIG. 4 is schematic in nature; and although not shown, the blades 70b may be held together as a unitary assembly by a resilient, elastic sleeve or other comparable component, which extends over their outer surfaces.

A still further mechanism for operating the access port expander is schematically illustrated in FIG. 5. In this embodiment, blades 70c are spread radially outward by actuation of a fluid drive system 146. The fluid drive system has a piston or actuator rod 60c sealingly mounted in an open end of a cylinder 150. Each of the blades 70c has radially extending drivers 156 that are sealingly mounted in, and extend through, the cylinder 150. The cylinder 150 has an internal cavity 152 filled with a fluid 154, for example, air, saline or any other fluid that can move the drivers 156 in response to a translation of the actuator rod 60c. As described in earlier embodiments, the upper end of the actuator rod 60c is threadedly engaged with the drive screw 44 of actuator 67; and therefore, rotation of the upper handle 40 causes the actuator rod 60c to move downward in the cylinder 150. That downward motion creates fluid forces against the ends 158 of the drivers 156, thereby moving the blades 70c in a radially outward direction as indicated by the arrow 160, that is, perpendicular to the actuator rod centerline or the blade lengths.

The access port expander 24 shown and described herein is effective to radially spread an access port 22, thus spreading and separating tissue laterally with respect to the incision site. Such a lateral spreading minimizes cutting of tissue, is generally parallel to a major surface of the skin and thus, tends to minimize and reduce wound trauma. Further, such a lateral spreading minimizes separating of tissue in a direction generally perpendicular to a major surface of the skin, that is, in a direction of penetration of the access port, which may increase wound trauma.

While the present invention has been illustrated by a description of various preferred embodiments and while these embodiments have been described in some detail, it is not the intention of the Applicant to restrict or in any way limit the scope of the appended claims to such detail. Additional advantages and modifications will readily appear to those skilled in the art. In the embodiments shown and described herein, a plurality of elongated blades are moved from a contracted position to an expanded position in response to operation of an actuator having an actuator rod in an operable relationship with the blades. Although several embodiments of the actuator are shown and described, other comparable embodiments are also known, for example, the upper and lower handles 40, 48 may contain an electric motor that imparts a rotation to the drive screw 44. A switch that is built into the handles or remotely actuated may operate the motor, and the motor may be powered by a battery or connected to an external power source.

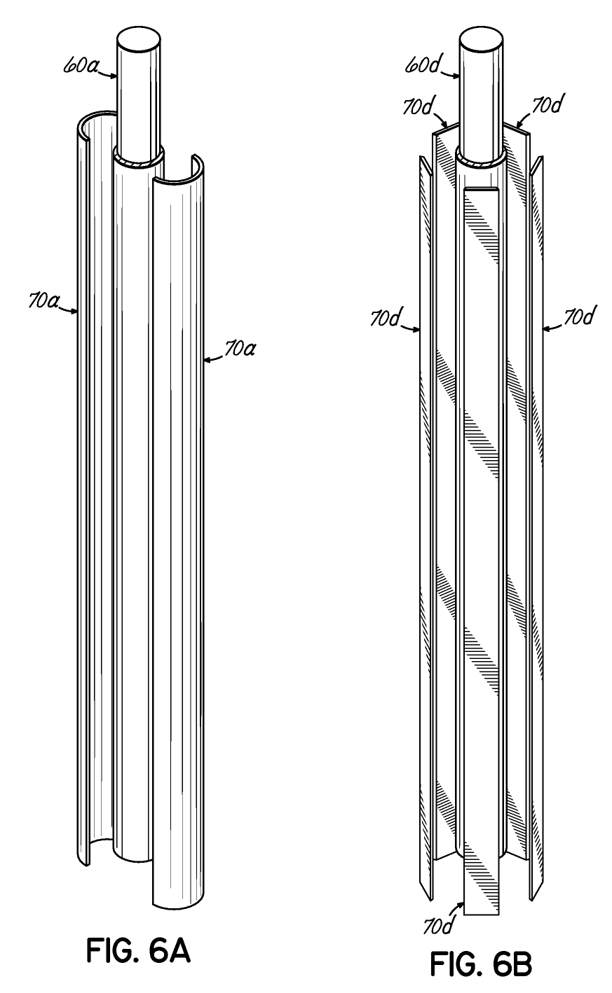

In the embodiments of FIGS. 1 and 2, the access port expander 24 has three blades; but in the embodiments of FIGS. 3-5, two blades are used. The number of blades often depends on the size of the access port expander 24 and its application. For example, for the smallest access port expanders, generally only two blades are used because the physical size does not permit more blades. However, as the size of the access port expander increases, as shown in FIG. 6B, more blades 70d may be mounted around an actuator rod 60d.

Further, in the embodiments shown and described, the blades 70 have a curved cross-sectional profile. The degree of curvature varies depending on the number of blades 70 used and the desired final cross-sectional profile desired. For example, most often, the blades 70 have a cross-sectional profile that results in the access port expander 24 providing a port having a generally circular cross-sectional profile when the blades are in their expanded state. However, in an alternative embodiment, as shown in FIG. 6A, if two blades are used, which have semicircular cross-sectional profiles, then moving the blades to an expanded state provides an opening having an elongated cross-sectional profile. In still further embodiments, as shown in FIG. 6B, the cross-sectional profile of the blades can be linear, which, in their expanded states, provides an access port having a cross-section shape similar to a polygon.

Further, in the embodiments shown and described herein, the access port expander 24 deploys the blades 70 in a direction generally perpendicular to the blade lengths and an axial centerline of the actuator rod 60, thereby expanding the access port 22 uniformly to form an access port 22 having a generally cylindrical shape. In some applications, it may be desirable for the access port 22 to have angled exterior walls in which one end of the access port 22 is larger or more expanded than its other end. This can be readily accomplished by changing the lengths of the drive links 72. For example, referring to FIG. 3, if the upper pair 162 of drive links 72a is made longer than the lower pair 164 of drive links 72a, the access port expander 24 will expand the access port 22 in a tapered form such that the lower end near the surgical site 26 has a smaller opening than the opposite end near the skin surface 36. Alternatively, by making the drive link upper pair 162 shorter than the drive link lower pair 164, the access port expander 24 will expand the access port 22 in a generally conical form such that the opening near the skin surface 36 is smaller than the opening near the surgical site 26.

Also, in the embodiments shown and described, the access port expander 24 is used to dilate or expand an access port 22; however, in alternative embodiments, the access port expander 22 may be used to dilate or expand a simple opening or unlined hole in the tissue without using the access port 22.

While the invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicants' general inventive concept.

What is claimed is:

1. A surgical access port expander for expanding an access path: between an incision site and a surgical site during a surgical procedure, comprising:
an actuator comprising an actuator rod; and
a plurality of blades having respective lengths and being movable in response to motion of the actuator rod moving in a direction substantially parallel to the respective lengths, each of the blades being movable in a direction substantially perpendicular to a respective length from a contracted position to an expanded position.

2. The surgical access port expander of claim 1 wherein the actuator further comprises:
a rotational component;
a mechanism connected between the rotational component and the actuator rod for converting a substantially rotational motion of the rotational component into a substantially axial motion of the actuator rod.

3. The surgical access port expander of claim 1 wherein the actuator further comprises:
an upper handle;
a lower handle rotatably connected to the upper handle;
a shaft having one end fixed to the upper handle and extending through the lower handle; and
the actuator rod being threadedly coupled to an opposite end of the shaft, the actuator rod being mounted in the lower handle to prohibit rotation but permit an axial translation, and thus, the actuator rod the actuator being movable in an axial direction in response to rotation of the upper handle.

4. The surgical access port expander of claim 1 wherein each of the plurality of blades comprises a link having one end pivotally connected to a respective blade and an opposite end pivotally connected to the actuator rod.

5. The surgical access port expander of claim 1 wherein each of the plurality of blades comprises at least two links, each of the links having one end pivotally connected to a respective blade and an opposite end pivotally connected to the actuator rod.

6. The surgical access port expander of claim 5 wherein each of the links has a different length.

7. The surgical access port expander of claim 1 wherein the plurality of blades comprised one of two, three, four, five blades and more than five blades.

8. The surgical access port expander of claim 1 wherein the plurality of blades comprises a generally circular cross-sectional profile.

9. The surgical access port expander of claim 1 wherein the plurality of blades comprises a generally noncircular cross-sectional profile.

10. The surgical access port expander of claim 1 wherein each of the plurality of blades comprises a generally curved cross-sectional profile.

11. The surgical access port expander of claim 1 wherein each of the plurality of blades comprises a generally linear cross-sectional profile.

12. A surgical access port expander for expanding an access path between an incision site and a surgical site during a surgical procedure, comprising:
an actuator comprising an actuator rod and operable to move the actuator rod in an axial direction;
a plurality of links, each of the links having one end pivotally connected to the actuator rod; and
a plurality of blades having respective lengths and mounted around the actuator rod generally in parallel with each other and the actuator rod, each of the plurality of blades being pivotally connected to opposite ends of two of the links, each of the of blades being movable from a contracted position toward an expanded position in response to the actuator moving the actuator rod axially in a direction substantially parallel to a respective blade length, and each of the blades moving in a direction substantially perpendicular to the respective blade length in response to axial motion of the actuator rod.

13. A method of using an access port expander during a surgical procedure to expand an access path between an incision site and a surgical site in a patient, the method comprising:
inserting a contracted access port expander through a collapsed expandable access port, the access port expander comprising an actuator having an actuator rod operably positioned with respect to a plurality of blades having respective lengths;
making an incision to create an incision site;
inserting the contracted access port expander and the collapsed expandable access port through the incision site until one end of the collapsed expandable access port is near the surgical site;
operating the actuator to move the actuator rod in a first direction substantially parallel to the respective lengths and simultaneously move the plurality of blades in a second direction substantially perpendicular to the respective lengths, the plurality of blades expanding radially outward to cause the expandable port expander to simultaneously expand outward in the second direction to an expanded state; and
removing the access port expander from the expandable access port to provide an expanded access path between an incision site and a surgical site to facilitate a surgical procedure.

14. The method of claim 13, further comprising:
inserting a guide wire through an incision site and toward a surgical site.
